(19) United States
(12) Patent Application Publication
(10) Pub. No.: US 2004/0000596 A1
(43) Pub. Date: Jan. 1, 2004

(54) Scent dispensing device

(76) Inventor: Lee Cuthbert, San Francisco, CA (US)

Correspondence Address: WILLIAM L. PARADICE, III, 425 CALIFORNIA STREET, SUITE 900, SAN FRANCISCO, CA, 94104, US

(21) Appl. No.: 10/378,450

(22) Filed: Mar. 3, 2003

Related U.S. Application Data
(63) Continuation-in-part of application No. 10/185,548, filed on Jun. 28, 2002.

(51) Int. Cl. A61L 9/04

(52) U.S. Cl. 239/34

(57) ABSTRACT

A scent dispensing device includes a hollow cavity defined by a resilient material configured to form a substantially flat packet, a porous and absorbent scent emitting material disposed within and free to move within the cavity, the scent emitting material retaining a scent which is imparted to air within the cavity, and an opening formed in the resilient material, the opening allowing air within the cavity to escape the packet during a compression of the packet and allowing air outside the packet to enter the packet when the compression terminates.

### CROSS-REFERENCE TO RELATED APPLICATION

This application is a continuation-in-part, and claims the benefit under 35 USC §120, of co-pending and commonly owned U.S. patent application Ser. No. 10/185,548 entitled “Scent Dispensing Packet” filed on Jun. 28, 2002, and incorporated herein by reference.

### FIELD OF INVENTION

The present invention relates generally to scent dispensers and specifically to an inexpensive and compact scent dispensing device.

### DESCRIPTION OF RELATED ART

It has become common practice to distribute fragrance samples to potential customers by inserting scent strips in magazines or mailings. The scent strip process involves microencapsulating a fragrance and adhering it with glue to a printed magazine or mailing insert. The fragrance is released when the microcapsules are burst. This is usually accomplished by separating two sheets containing the microcapsules that have been temporarily adhered to one another. These burst microcapsules emit a scent because they are exposed to the air. This limits the reuse of this system as a sampling vehicle because the fragrance material cannot be easily protected from evaporation. Fragrance material is very volatile and evaporates quickly.

For instance, U.S. Pat. Nos. 5,188,236 (Sayers. et al.), 5,391,420 (Bootman, et al.), and 4,817,860 (Shapiro) all disclose releasing fragrance samples through bursting microcapsules adhered to a substrate paper or cardboard backing. U.S. Pat. No. 5,419,958 (Charbonneau) discloses a process for preventing unwanted escape of fragrance material prior to fracturing of the microcapsules. However, in all of this prior art, the problem of quick evaporation of the fragrance material once the microcapsules are burst still exists.

This problem also exists with U.S. Pat. No. 6,123,221 (Simpson) which discloses a process to deliver folded scented coupons in a dispenser. These coupons contain microcapsules that fracture as the coupons are removed from the dispenser. Once the coupons have been activated, the scent quickly evaporates.

It has become standard practice in the cosmetics industry to mail or otherwise distribute samples of fragrances to potential customers. Frequently such samples are distributed in the form of “vial-on-cards”, which usually comprise a glass or plastic vial fastened to a cardboard backing. Another known means for packaging a unit dosage cosmetic sample or fragrance is the soft gelatin capsule. Similar in function to plastic or glass vials, these “soft gel” capsules contain a fluid sample of the fragrance.

These types of fragrance samples are awkward to use because they are so small. The fragrance must be applied to the skin to experience its intended scent because it is otherwise too concentrated. Once a fragrance is applied to the skin it begins to evaporate and can wear off in a matter of hours. U.S. Pat. No. 5,535,885 (Daniel; et al.) discloses a means to produce a unit-dose container that can survive the rigors of shipment through the mails or inclusion in printed material but it still does not solve the inherent problem of ease of use and reduction of evaporation once the fragrance has been released from the container.

Devices that do not expose volatile fragrance oils or burst microcapsules directly to the surrounding air can diminish the rate of scent evaporation. This is accomplished by enclosing scented elements in a sack or “sachet”. The purpose of these sacks is to slowly deliver a pleasant scent to an area, usually to mask an unpleasant odor. U.S. Pat. Nos. 5,503,332 (Glenn), 4,306,892 (Atalie, et al.), and 4,854,501 (Ricci) all disclose fragrance holders for scenting the surrounding area by permeation of scents into the air. These sacks are not effective promotion vehicles for delivering scent samples because they are not convenient to distribute through the mail due to their size and shape. In addition, they have the disadvantage of delivering scent to a wide area thereby causing the scent to linger for an extended period of time.

Therefore, there is a need for better device and method for delivering a desired scent to a consumer.

### BRIEF DESCRIPTION OF THE DRAWINGS

The features and advantages of the present invention are illustrated by way of example and are by no means intended to limit the scope of the present invention to the particular embodiments shown, and in which:

FIG. 1 is a perspective front view of a scent dispensing packet at an equalized air pressure state;

FIG. 2 is a front cross-sectional view of the scent dispensing packet of FIG. 1 showing a scent emitting material housed therein;

FIG. 3 is a side view of the scent dispensing packet of FIG. 1 at an equalized air pressure state;

FIG. 4 is a side view of the scent dispensing packet of FIG. 1 being squeezed by a user;

FIG. 5 is a side view of the scent dispensing packet of FIG. 1 returning to its equalized air pressure state;

FIG. 6 is a perspective front view of the scent dispensing packet prior to assembly;

FIG. 7 is a perspective front view of the scent dispensing packet with a purchase incentive affixed;

FIG. 8 is a perspective front view of the scent dispensing packet affixed to an interior of a greeting card;

FIG. 9 is a perspective front view of the scent dispensing packet affixed to an advertising or promotion card;

FIG. 10 is perspective front view of an embodiment of the scent dispensing packet having a sealant;

FIG. 11 is perspective front view of the scent dispensing packet containing within a protective sleeve;

FIG. 12 is a perspective front view of the scent dispensing packet in accordance with another embodiment of the present invention;

FIG. 13 is a perspective front view of the scent dispensing packet in accordance with another embodiment of the present invention; and

FIG. 14 is a perspective front view of another embodiment of the scent dispensing packet.

Like reference numerals refer to corresponding parts throughout the drawing figures.

### DETAILED DESCRIPTION

The present invention is discussed below with respect to several exemplary embodiments for simplicity only. It is to be understood that those skilled in the art will, after reading this disclosure, be able to implement the present invention in other ways that are not specifically shown herein. Accordingly, the present invention is not to be construed as limited to specific examples described herein but rather includes within its scope all embodiments defined by the appended claims.

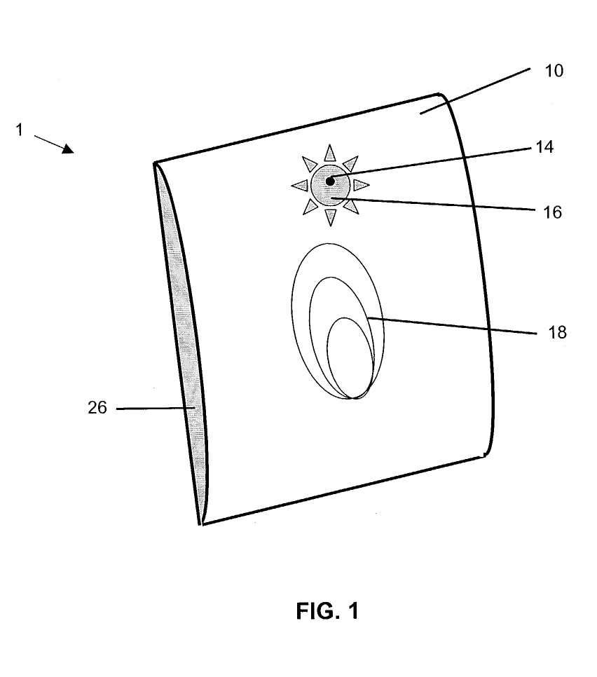

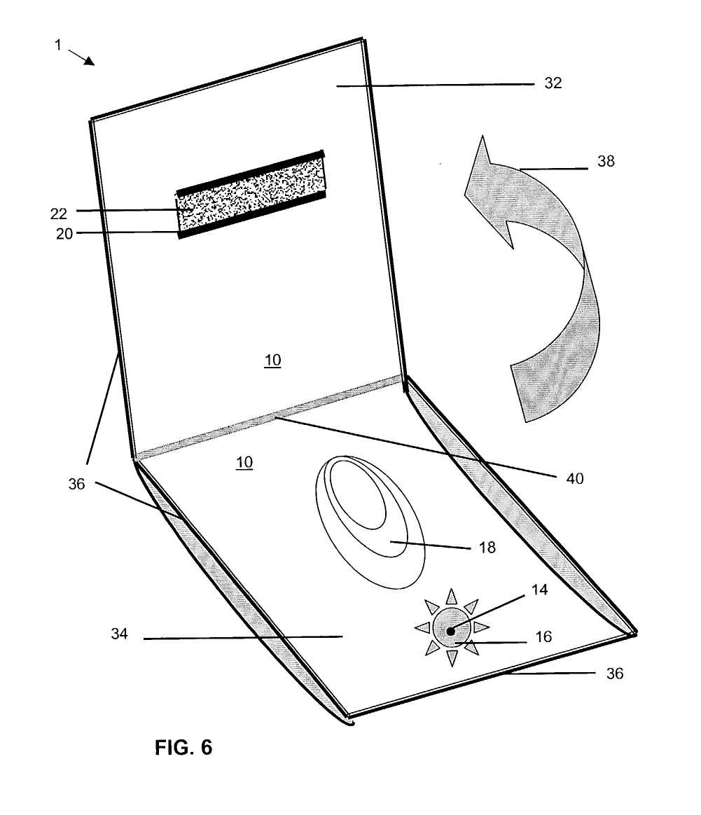

FIG. 1 shows a scent dispensing packet 1 in accordance with one embodiment of the invention. The packet 1 includes an outer wall 10 made of plastic, foil or foil laminate or any other impermeable, flexible, thin material that can be sealed or molded into the shape of the packet. For some embodiments, outer wall 10 is a single piece of material that is folded and sealed to form the packet 10 as described below, for example, with respect to FIG. 6. When folded and sealed to form the packet 1, outer wall 10 defines a cavity 26 within the packet 1. An airflow valve 16 having an opening 14 formed therein is provided on a front surface of packet 1. The opening 14 and valve 16 may be made of plastic or any other durable, moldable material. For some embodiments, the opening 14 and valve 16 may be integrated within the outer wall 10. Together, the opening 14 and valve 16 allow air to pass into and out of the cavity 26 within the packet 1. For some embodiments, a design element 18 may be provided on a exterior surface of outer wall 10 to indicate to a user where to put his/her thumb on the packet 1 for activation or squeezing.

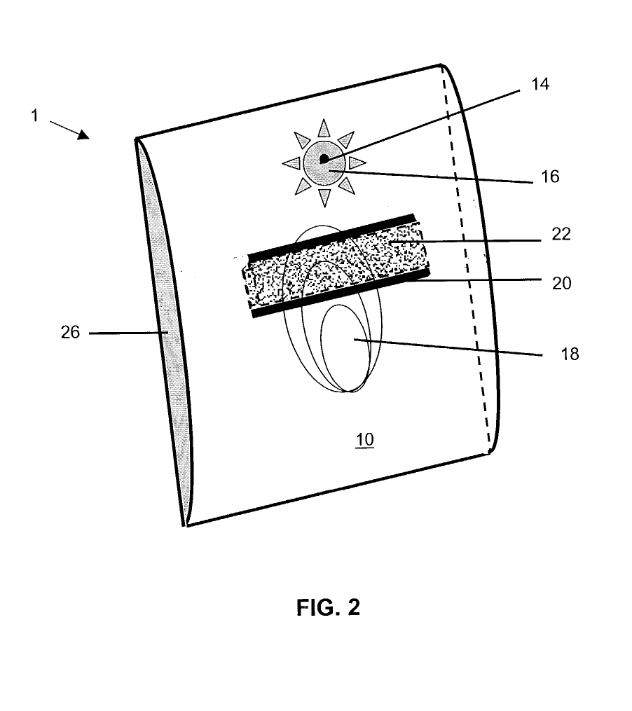

FIG. 2 shows the scent dispensing packet 1 containing a scent emitting material holder 20 and a scent emitting material 22. The holder 20 may be molded into an interior surface of the outer wall 10 of the packet 1. The holder 20 holds the scent emitting material 22 in place within the packet 1. The scent emitting material 22 is made of a porous material capable of absorbing and retaining a desired scent or fragrance. Further, the scent emitting material 22 is capable imparting the retained scent to the air within the cavity 26. The desired scent or fragrance can be imparted to the scent emitting material 22 by immersing, spraying or otherwise exposing the porous material 22 to a solution containing the desired scent or fragrance. The scent emitting material 22 may be any suitable material that retains a desired scent. These materials include, for example, cellulose, polyester, polyolefin, cotton, porous plastics, fiberglass, and natural sponges.

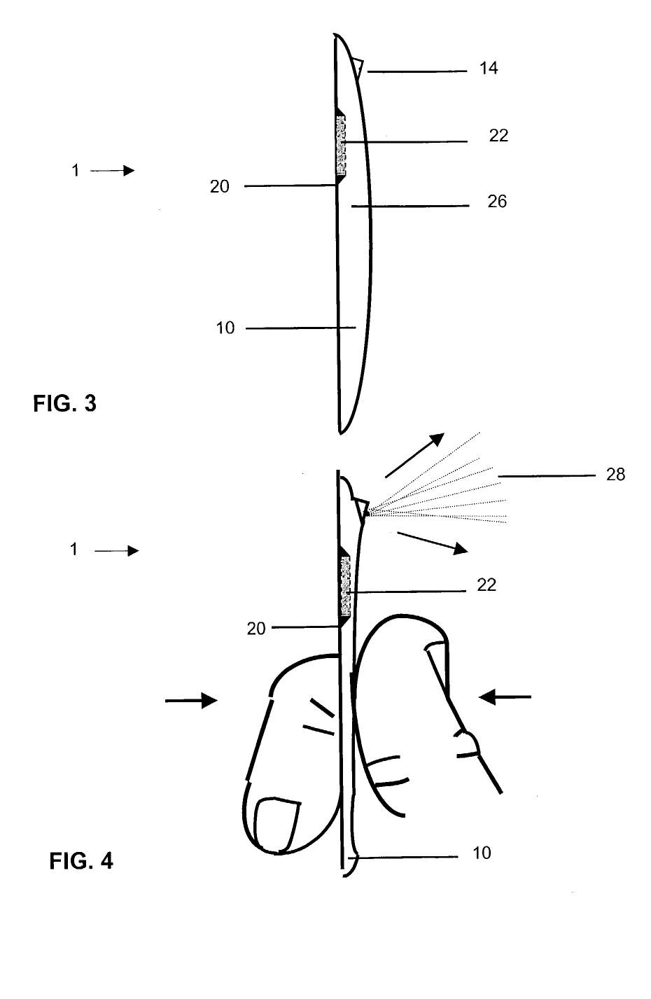

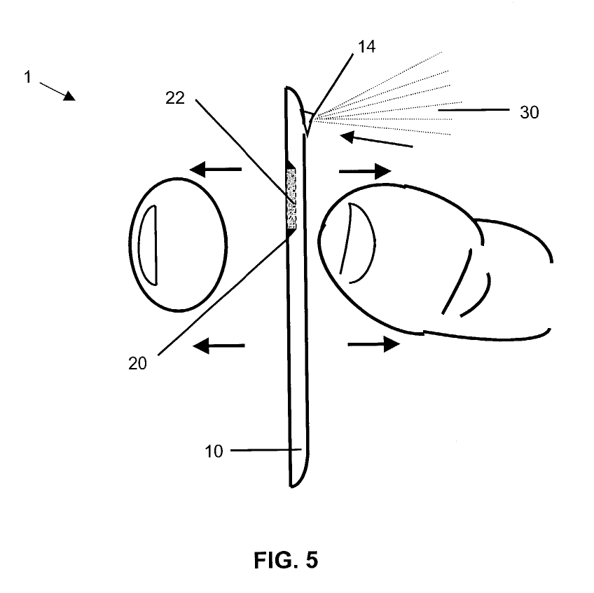

FIGS. 3-5 illustrate operation of the scent dispensing packet of the present invention. FIG. 3 shows the packet 1 is its initial state, where the cavity 26 is at its maximum (steady state) volume. When the packet 1 is compressed (e.g., by a user's thumbs), as shown in FIG. 4, the air within cavity 26 which surrounds the scent emitting material 22 is forced to the outside environment (as indicated by exiting air 28) through the opening 14 in the packet wall 10, thereby imparting the desired scent to the outside environment, for example, to be smelled or otherwise sampled by the user. Then, when the user's thumb are released from the packet 1, as shown in FIG. 5, a vacuum created within the packet 1 draws air 30 from the outside environment into packet cavity 26 via the opening 14 in the valve 16, thereby filling the cavity 26 with fresh air 30 and returning the packet 1 to its original steady state. Thereafter, the scent emitting material 22 imparts its scent to the fresh air within cavity 26, thereby readying the packet 1 for subsequent use.

FIG. 6 shows the packet 1 before it has been fully assembled, in which the scent emitting material 22 is affixed to an interior surface of a first portion 32 of outer wall 10. For some embodiments, the scent dispensing packet 1 is manufactured from a singular piece of material by molding the material in a flat, open position using any well-known injection or stamp molding process. After the molding process, the scent emitting material 22 is placed into the scent emitting material holder 20, which may either be attached to or integrated within outer wall material 10. Then, packet wall portion 32 is folded at a flexible hinge 40 and closed in the direction of arrow 38 to mate with opposing packet wall portion 34. The hinge 40 may be molded into the design of the packet wall 10. Interlocking grooves 36 molded into three edges of the packet wall 10 maintain the packet 1 in its closed position. For other embodiments, opposing wall portions 32 and 34 may be sealed together along peripheral edges thereof using a suitable sealant such as, for example, glue or other adhesive.

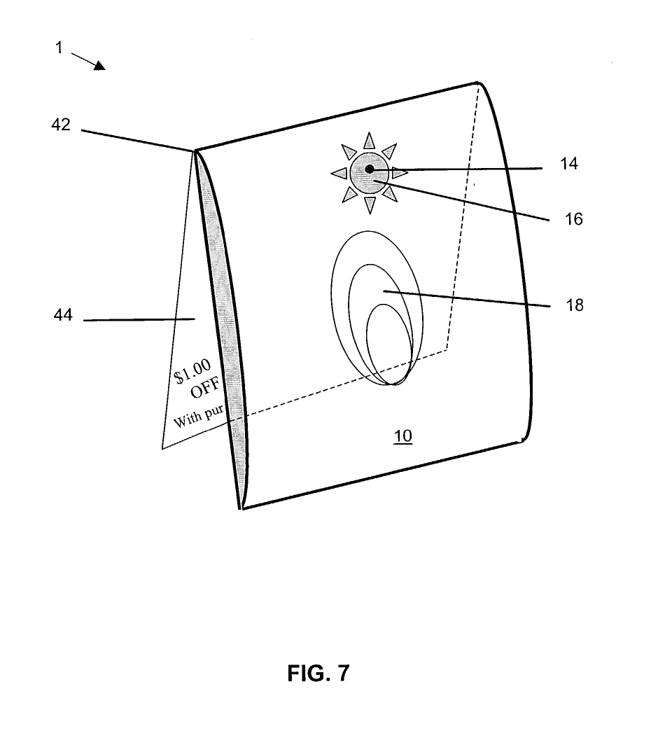

FIG. 7 shows the scent dispensing packet 1 with a purchase incentive 44 attached to a top edge 42 of the packet. The incentive 44 may be affixed to the packet using glue or other adhesive.

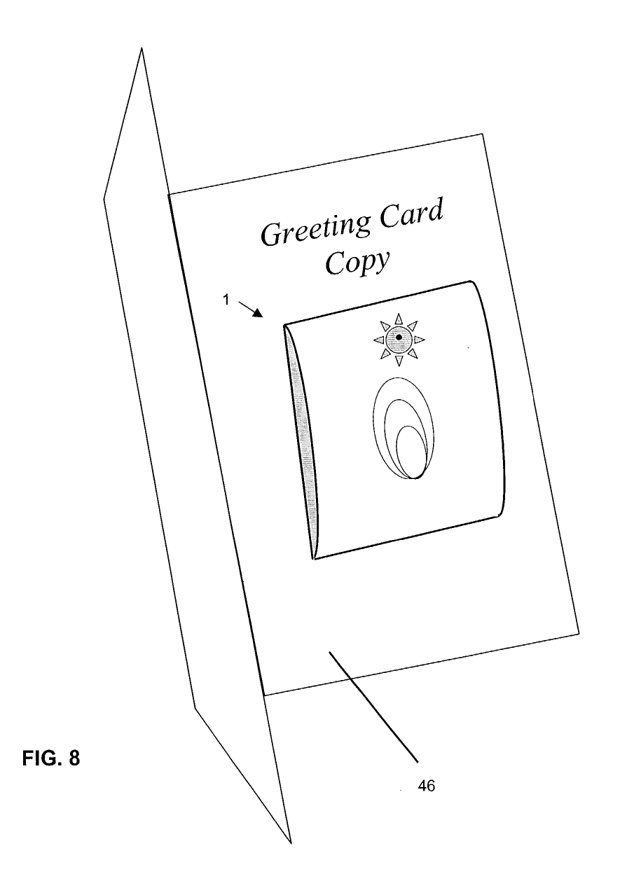

FIG. 8 shows a greeting card 46 into which the packet 1 may be affixed, for example, using glue or other adhesive.

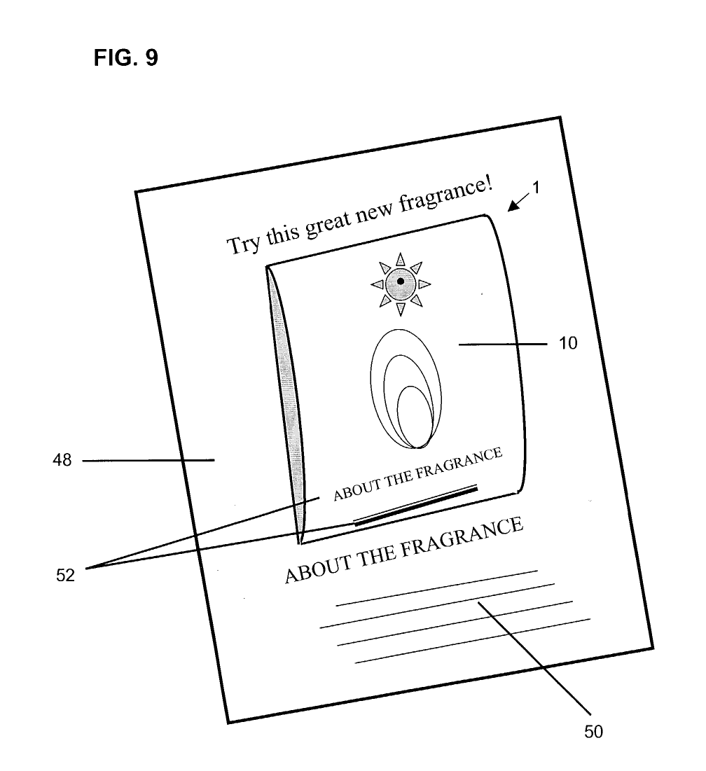

FIG. 9 shows an advertising or promotion card 48 onto which the packet 1 may be affixed. Marketing information 50 may also be provided on the card 48.

As described above and shown in the figures, scent dispensing packet 1 is small, lightweight, inexpensive, and reusable. The packet 10 may be any suitable size. For some embodiments, the packet is substantially flat and adapted to be provided within a greeting card, as illustrated in FIG. 8. For one embodiment, the packet 1 has height of approximately 6 centimeters (cm), a width of approximately 3 cm, and a thickness (e.g., as defined by the cross-section of cavity 26) of approximately 0.5 cm.

The size and configuration of packet 1 provides other advantages. First, because the packet 1 is substantially flat, graphics 52 and other information may be easily printed onto exterior portions of the outer wall 10, as shown in FIG. 9. In this manner, the packet 1 may be readily used as a sampling device for consumer products having a scent. For example, a manufacturer or retailer of perfume may impart the perfume into the scent emitting material 22 of the packet 1, provide graphics 52 identifying the particular perfume and/or other information, and then provide the packet 1 at retail outlets so that consumers may easily sample the perfume without opening any of the actual perfume containers. In this manner, retailers may allow consumers to easily sample the perfume's scent without designating one or more of the actual perfume products as samples, thereby reducing marketing costs.

Of course, although discussed above in the example of sampling perfumes, packets 1 may be used to allow consumers to sample the scent of virtually any product including, for example, shampoos, deodorants, skin care products, foods, beverages, and so on.

Second, because the packet 1 is small, inexpensive, and portable, retailers may allow consumers to take with them several packets 1 containing various scents corresponding to different products for further sampling and/or to share with friends or family. In this manner, the packet 1 may provide a competitive advantage to such retailers by allowing consumers to distribute perfume samples to other perspective buyers. For example, packets 1 are well-suited for door-to-door sales persons who sell perfumes or other scented products.

Third, the packet 1 may be inserted into a greeting card (as illustrated in FIG. 8) and easily mailed to one or more recipients without any special packaging or without increased mailing costs. In contrast, for example, prior art vials containing a scent or fragrance which are delivered through the mail must be carefully packaged to prevent damage to the vials, thereby increasing shipping and packaging costs.

Further, in contrast to some prior art scent dispensers, embodiments of the packet 1 deliver air imparted with a scent from the scent emitting material 22 to the user for sampling without applying the source fragrance to the user's skin. This may be preferable for consumers who do not wish to spray a scented chemical mist onto their skin. In addition, because the packet 1 dispenses air imparted with a scent, rather than spraying a scented chemical mist toward the user, the packet 1 uses less scent during use, thereby maximizing the useful life of the packet.

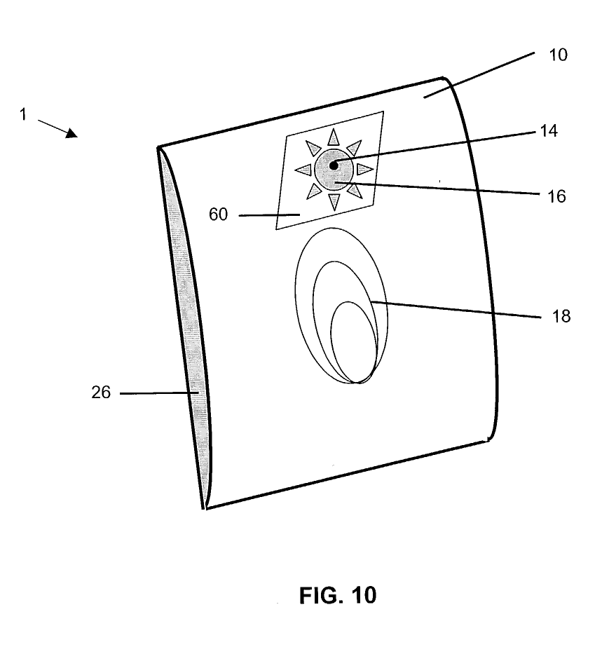

Referring to FIG. 10, the air opening 14 for the scent dispensing packet 1 may be sealed with a suitable sealant 60 after the packet 1 is fabricated to prevent the scent retained by the scent emitting material 22 from escaping the packet 1 prior to use, thereby prolonging the shelf life of the packet. The sealant 60 may be any suitable material including, for example, tape or other adhesive material.

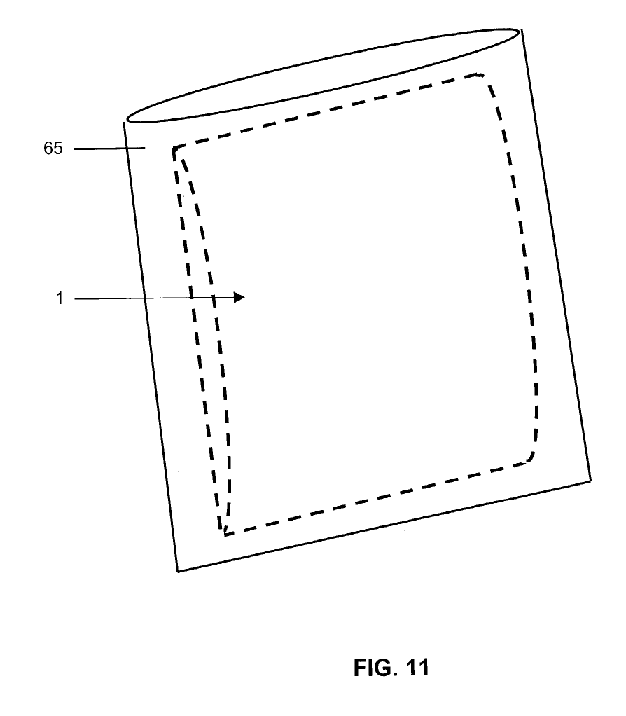

For some embodiments, the packet 1 may be slidably disposed in a protective sleeve 65, as illustrated in FIG. 11. The protective sleeve 65, which may be any suitable material including, for instance, cardboard, laminate, plastics, or the like, protects the packet 1 from environmental forces (e.g., air, water, bending, and so on). In addition, graphics or other identifying information (not shown for simplicity) may be included on the front and/or back surfaces of protective sleeve 65 to provide information to consumers about the scent retained therein and/or information about the scent's manufacturer or retailer. For some embodiments, the graphics on the protective sleeve 65 may include decorative arts.

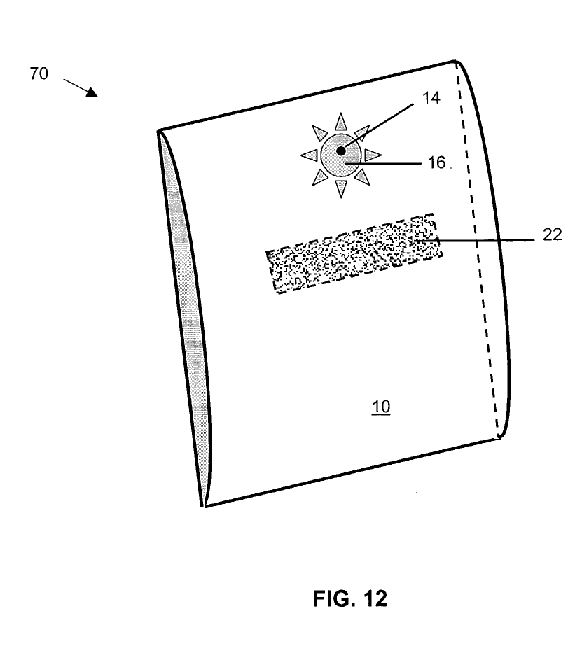

FIG. 12 shows a packet 70 that is another embodiment of the present invention. Packet 70 is similar to packet 1 described above, except that the holder 20 is omitted, thereby allowing the scent emitting material 22 to be slidably disposed within cavity 26. Thus, by allowing the scent emitting material 22 to move freely within cavity 26, the air within cavity 26 may be more effectively exposed to, and thus imparted with, the scent retained by the scent emitting material 22.

A desired scent can be imparted to the scent emitting material 22 either before or after the packet is manufactured. For one embodiment, the scent emitting material 22 is immersed in, sprayed with, or otherwise exposed to the desired scent before the packet is constructed, as described above. For other embodiments, the scent emitting material 22 may first be inserted into the packet, without being scented, and then the packet closed and sealed as described above. Thereafter, a desired scent may be injected into the cavity 26 within the packet, for example, by injecting a needle containing the desired scent into the scent emitting material 22 via opening 14. In this manner, consumers, retailers, or manufacturers provided with unscented packets may be able to impart their own scents into the packets, thereby allowing the packets to be customized by such consumers, retailers, or manufacturers.

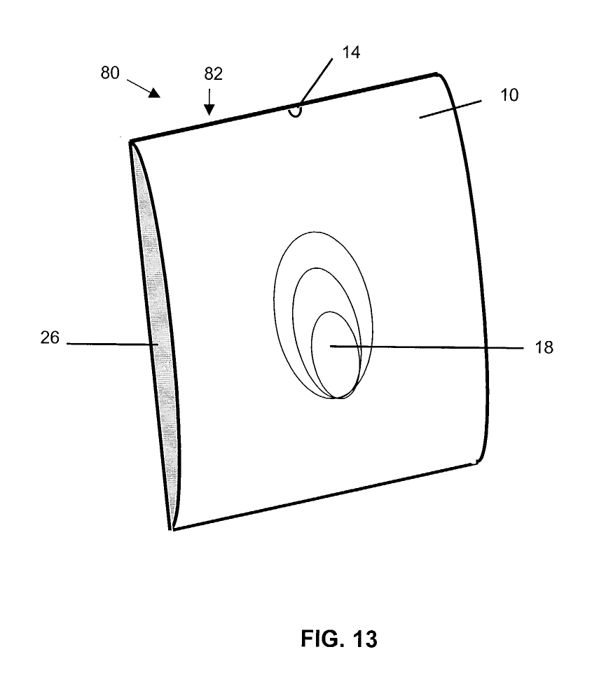

For other embodiments, the valve 16 may be omitted, and the opening 14 may be located on a suitable edge of the packet. For example, FIG. 13 shows a packet 80 that is another embodiment in accordance with the present invention. Packet 80 is similar to packets 1 and 70, except that the opening 14 is located on an upper edge 82 of the packet 80. For some embodiments of FIG. 13, the scent emitting material 22 may be held in place by the holder 20 (not shown for simplicity). For other embodiments of FIG. 13, the holder may be eliminated, thereby allowing the scent emitting material 22 to be slidably disposed within the packet.

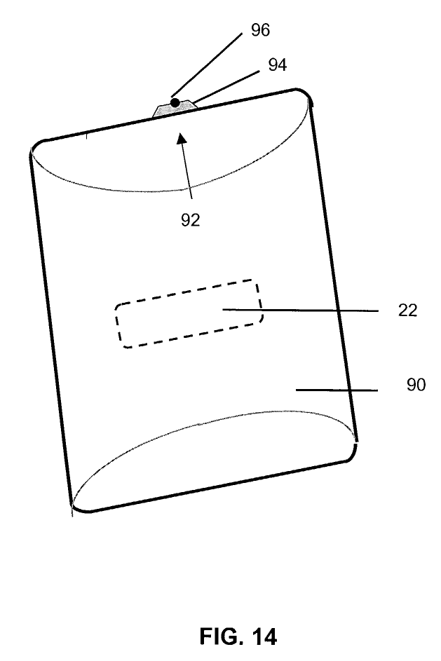

FIG. 14 shows a scent emitting bottle 90 in accordance with another embodiment of the present invention. Bottle 90 is constructed a resilient, flexible plastic or similar material using well known injection molding techniques, and contains the scent emitting material 22. Bottle 90 has an opening 92 formed in a top surface thereof. The opening 92 contains grooves (not shown for simplicity) onto which a top 94 having a small opening 96 may be screwed. The scent emitting material 22 described above may be inserted into the bottle 90 through the opening 92 (with the top removed). Thereafter, during use (with the top 94 screwed onto opening 92, the bottle 90 may be squeezed to cause scent laden air within the bottle to exit through top opening 96, thereby imparting the scent to the external environment for sampling. After use, the bottle's resilient walls return to their original form, which creates a vacuum that causes fresh air to be drawn into the bottle, after which the scent emitting material 22 imparts the scent to the fresh air for subsequent use.

For such embodiments, the scent emitting material 22 may be provided separately from the bottle and packaged in a foil casing (not shown) to prevent evaporation prior to use. For other embodiments, the scent emitting material 22 may be covered with a suitable membrane that protects a user's hands from the scent while allowing air to pass therethrough. For other embodiments, the scent emitting material 22 may contain micro-encapsuled scent oil.

While particular embodiments of the present invention have been shown and described, it will be obvious to those skilled in the art that changes and modifications may be made without departing from this invention in its broader aspects and, therefore, the appended claims are to encompass within their scope all such changes and modifications as fall within the true spirit and scope of this invention.

What is claimed is:

1. A scent dispensing device comprising:
a hollow cavity defined by a resilient wall which forms a substantially flat packet;
a porous and absorbent scent emitting material freely disposed within the cavity, the scent emitting material retaining a scent that is imparted to air within the cavity; and
an opening formed in the resilient wall, wherein the scented air within the cavity is forced through the opening and outside the packet when the packet is compressed.

2. The device of claim 1, wherein the scented air that is forced outside the packet is adapted to be smelled by a user.

3. The device of claim 2, wherein the scented air is not a chemical mist.

4. The device of claim 1, wherein the opening allows fresh air from outside the packet to enter the cavity when the packet is de-compressed.

5. The device of claim 1, wherein packet is adapted to fit within a user's hands and adapted to be squeezed by the user's thumbs.

6. The device of claim 1, wherein the packet is adapted to fit within a folded greeting card.

7. The device of claim 1, wherein the scent emitting material consists of one of the following: cellulose, polyester, polyolefin, cotton, porous plastics, fiberglass, and natural sponge.

8. The device of claim 1, wherein the packet has a height of approximately 6 centimeters, a width of approximately 3 centimeters, and a thickness of approximately one-half centimeter.

9. The device of claim 1, wherein resilient wall is a single, rectangular piece of molded material having interlocking grooves on three edges thereof.

10. The device of claim 1, wherein the opening is formed along an edge of the packet.

11. The device of claim 1, further comprising a removable sealant provided over the opening.

12. The device of claim 1, further comprising:
graphics printed on an exterior surface of the resilient wall.

13. The device of claim 1, further comprising:
a protective into which the packet is slidably disposed.

14. A scent dispensing device comprising:
a hollow cavity defined by a resilient and squeezable bottle;
a porous and absorbent scent emitting material freely disposed within the cavity, the scent emitting material retaining a scent that is imparted to air within the cavity; and
an opening formed in the bottle, the opening allowing scented air within the cavity to escape from the device when the bottle is compressed, and allowing fresh air to enter the cavity when the bottle is de-compressed.

15. The device of claim 14, wherein the scented air that is forced outside the bottle is adapted to be smelled by a user.

16. The device of claim 15, wherein the scented air is not a chemical mist.

17. The device of claim 14, wherein bottle is adapted to fit within a user's hands and adapted to be squeezed by the user's thumbs.

18. The device of claim 14, wherein the scent emitting material consists of one of the following: cellulose, polyester, polyolefin, cotton, porous plastics, fiberglass, and natural sponge.

19. A method of dispensing a scent, comprising:
providing a substantially flat packet defining a cavity containing a scent emitting material that is imparted with the scent;
squeezing the packet to force air imparted with the scent from the cavity through an opening in the packet to outside the packet so as to be detectable by smelling; and
releasing the packet to force fresh air from outside the packet through the opening to the cavity, the fresh air within the cavity subsequently imparted with the scent from the scent emitting material.

20. The method of claim 19, wherein the packet does not dispense a chemical mist.

21. The method of claim 19, wherein packet is adapted to fit within a user's hands and adapted to be squeezed by the user's thumbs.

22. The method of claim 19, wherein the packet is adapted to fit within a folded greeting card.

23. The method of claim 19, wherein the scent emitting material comprises a porous material.

24. The method of claim 23, wherein the porous material consists of one of the following: cellulose, polyester, polyolefin, cotton, porous plastics, fiberglass, and natural sponge.
